(19) United States
(12) Patent Application Publication
(10) Pub. No.: US 2019/0087906 A1
(43) Pub. Date: Mar. 21, 2019

(54) REMOTE PROCESSING OF ANOMALOUS HEALTH OR LIFE SENSOR DATA

(71) Applicant: State Farm Mutual Automobile Insurance Company, Bloomington, IL (US)

(72) Inventors: Brian Mark Fields, Normal, IL (US); Duane Christiansen, Normal, IL (US)

(73) Assignee: State Farm Mutual Automobile Insurance Company, Bloomington, IL (US)

(21) Appl. No.: 16/108,952

(22) Filed: Aug. 22, 2018

Related U.S. Application Data
(60) Provisional application No. 62/560,909, filed on Sep. 20, 2017.

(51) Int. Cl. G06Q 40/08; G16H 50/30; G16H 10/65; G16H 15/00; H04W 4/38

(52) U.S. Cl. CPC G06Q 40/08; G16H 50/30; H04W 4/38; G16H 15/00; G16H 10/65

(57) ABSTRACT

A system and computer-implemented method for processing large amounts of sensor data, in which anomalous data is identified and transmitted in real time to a remote location for processing, and non-anomalous data is stored locally for later transmission during an off-peak time. In an insurance implementation, a local device receives sensor data, and identifies and transmits the anomalous data, while a remote server receives and analyzes the transmitted anomalous data, and based at least thereon, recommends an insurance premium. The insurance premium may be for vehicle, property, or health or life insurance. The anomalous data may be defined objectively or subjectively, and may be identified by an artificial intelligence tool. The remote server may generate a report, which may include a score, and transmit the report to the local device for display. The remote server may base the report and the insurance premium on the combined anomalous and non-anomalous data.

### RELATED APPLICATION

The current patent application is a non-provisional application which claims priority benefit to U.S. Provisional Application No. 62/560,909, entitled “SYSTEM AND COMPUTER-IMPLEMENTED METHOD FOR MULTI-STAGE PROCESSING OF SENSOR DATA”, and filed Sep. 20, 2017. The earlier-filed provisional application is hereby incorporated by reference in its entirety into the current patent application.

### FIELD OF THE DISCLOSURE

The present disclosure generally relates to systems and methods for processing data, and more particularly, to a system and computer-implemented method for processing large amounts of sensor data, wherein anomalous data is identified as such and transmitted to a remote location for processing, and non-anomalous data is pre-processed and temporarily stored locally for later transmission to the remote location and reconciliation with the anomalous data.

### BACKGROUND

Pay-as-you-drive systems, such as State Farm's Drive Safe & Save™ system, allow insurance providers to better assess insurance risks and reward proven safe drivers with lower premiums. One way to participate in such programs is through an electronic device that plugs into a vehicle's on-board diagnostic (OBD-II) port and records such relevant information as acceleration, turning, and braking. Another way is to make use of a telematics-based subscription service that records such information as mileage travelled. In still another way, drivers in California can self-report their mileage.

Telematics-based mobile applications allow the insurance industry to better match the behaviors of customers with appropriate insurance premiums. Typically, a mobile phone or other local device records data from sensors (e.g., speed, acceleration, turning, braking, geographic location) at a predefined interval (e.g., twenty times per second) and transmits the data to a remote server for processing and analysis. The server produces a summary or other report, and transmits the summary or other report back to the device for viewing by the customer.

However, network calls require large amounts of battery power and consume data from customer data plans, data storage is expensive with regard to both hardware and maintenance, data processing is greatly slowed by hundreds of thousands of users sending large amounts of data, and customer demand for real time data is rapidly increasing. During peak periods, a server receives such large amounts of real time data that processing, analysis, and summarization by the server normally require twenty-four to seventy-two hours to complete due to backlogs. All incoming data is queued for processing, so back-ups delay all customers from receiving feedback. In an exemplary implementation, telematics data may be transmitted from a vehicle to a remote server for processing, and during busy periods, one million or more devices may be transmitting data to a single server. Processing the data requires four to five minutes, which results in a one-day turnaround time for providing feedback to drivers about their trips.

### BRIEF SUMMARY

Embodiments of the present technology provide a system and computer-implemented method for processing large amounts of sensor data, wherein anomalous data is identified as such and transmitted in real or near real time to a remote location for processing, and non-anomalous data is pre-processed and temporarily stored locally for later transmission to the remote location and reconciliation with the anomalous data.

In a first aspect, a system may be configured to collect and process information relevant to setting an insurance premium. The system may broadly comprise a local electronic device and a remote server computer. The local electronic device may store and execute a software application configured to receive sensor data from one or more sensors, identify anomalous data in the sensor data as the anomalous data is received, and transmit the anomalous data as the anomalous data is identified. The remote server computer may be configured to receive the anomalous data transmitted by the software application, analyze the anomalous data, and recommend the insurance premium based at least in part on the anomalous data.

In a second aspect, a computer-implemented method may collect and process information relevant to setting an insurance premium. The computer-implemented method may broadly comprise the following. A local electronic device may receive sensor data from one or more sensors, identify anomalous data in the sensor data as the anomalous data is received, and transmit the anomalous data as the anomalous data is identified. A remote server computer may receive the transmitted anomalous data, analyze the anomalous data, and recommend the insurance premium based at least in part on the anomalous data.

Various implementations of any or all of the foregoing aspects may include any one or more of the following additional features. The local electronic device may be a smartphone, and the anomalous data may be transmitted over a wireless communication network to the remote server computer. The insurance premium may be for vehicle insurance, the sensor data may include speed, acceleration, turning, braking, cornering, stopping, phone usage, and/or location, and the anomalous data may include exceeding a speed limit, overly aggressive acceleration, turning, braking, cornering, and stopping, phone usage, driving and parking in high crime areas, non-use of a theft alarm, and/or significant impact or a collision. The insurance premium may be for property insurance, the sensor data may include doors or windows, security lighting, and thermal, audio, smoke, and/or security alarms, and the anomalous data may include open and unlocked doors or windows, non-use of security lighting, and/or activation of thermal, audio, smoke, or security alarms. The insurance premium may be for health or life insurance, and the sensor data may include heart rate and blood pressure, smoking, alcohol and drugs, exercise, and/or sleep, and the anomalous data may include high resting heart rate and blood pressure, smoking, use of alcohol or other illegal drugs or certain legal drugs, and/or inadequate sleep.

The anomalous data may be defined objectively and/or subjectively. An artificial intelligence tool may be used to identify the anomalous data. The server computer may be further configured to generate a report based on the anomalous data, wherein the report may include a score, and to transmit the report to the local electronic device for display. The report may be generated periodically or continuously.

The software application may be further configured to store non-anomalous data or a summary thereof, and to transmit the non-anomalous data or the summary thereof at an off-peak time. The remote server computer may be further configured to receive and analyze the non-anomalous data or the summary thereof, generate a report based on the anomalous data and the non-anomalous data or the summary thereof, wherein the report may include a score, and transmit the report to the local electronic device for display, and recommend the insurance premium based on both the anomalous data and the non-anomalous data or the summary thereof. The report may be generated periodically or continuously.

Advantages of these and other embodiments will become more apparent to those skilled in the art from the following description of the exemplary embodiments which have been shown and described by way of illustration. As will be realized, the present embodiments described herein may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed herein. It should be understood that each Figure depicts an embodiment of a particular aspect of the disclosed system and methods, and that each of the Figures is intended to accord with a possible embodiment thereof. Further, wherever possible, the following description refers to the reference numerals included in the following Figures, in which features depicted in multiple Figures are designated with consistent reference numerals. The present embodiments are not limited to the precise arrangements and instrumentalities shown in the Figures.

FIG. 1 is a block diagram of an embodiment of a system constructed in accordance with the present technology for processing large amounts of sensor data; and

FIG. 2 is a flowchart of an embodiment of a computer-implemented method which may be implemented by the system of FIG. 1.

The Figures depict exemplary embodiments for purposes of illustration only. One skilled in the art will readily recognize from the following discussion that alternative embodiments of the systems and methods illustrated herein may be employed without departing from the principles of the technology described herein.

### DETAILED DESCRIPTION

The following detailed description of embodiments of the invention references the accompanying figures. The embodiments are intended to describe aspects of the invention in sufficient detail to enable those with ordinary skill in the art to practice the invention. Other embodiments may be utilized and changes may be made without departing from the scope of the claims. The following description is, therefore, not limiting. The scope of the present invention is defined only by the appended claims, along with the full scope of equivalents to which such claims are entitled.

In this description, references to “one embodiment”, “an embodiment”, or “embodiments” mean that the feature or features referred to are included in at least one embodiment of the invention. Separate references to “one embodiment”, “an embodiment”, or “embodiments” in this description do not necessarily refer to the same embodiment and are not mutually exclusive unless so stated. Specifically, a feature, structure, act, etc. described in one embodiment may also be included in other embodiments, but is not necessarily included. Thus, particular implementations of the present invention can include a variety of combinations and/or integrations of the embodiments described herein.

The present technology may relate to, inter alfa, systems and methods for processing data. Broadly, certain embodiments of the present technology may provide a system and computer-implemented method for processing large amounts of sensor data, wherein anomalous data is identified as such and transmitted in real or near real time to a remote location for processing, and non-anomalous data is pre-processed and temporarily stored locally for later transmission to the remote location and reconciliation with the anomalous data. In exemplary implementations, the sensor data may be generated by telematics and concern the behaviors of insurance customers which may be relevant to determining appropriate insurance premiums and/or other insurance policy considerations.

Broadly, embodiments employ multi-stage processing, including initial device-side data processing backed by final server-side data processing, in order to improve the customer experience for telematics-based mobile applications, such as State Farm's Drive Safe & Save™ system. Multi-stage processing leverages the computational power of modern mobile devices to reduce the workload of the server-side processors, and thereby provides more efficient processing, faster turn-around times, and reduced costs for data-intensive telematics programs.

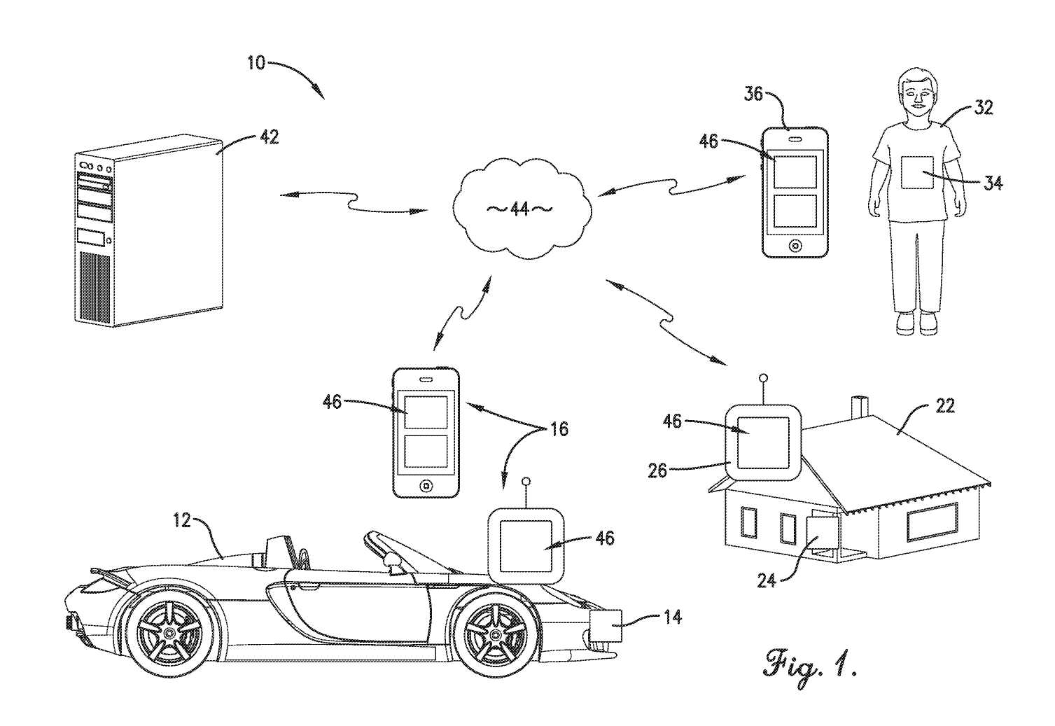

Referring to FIG. 1, an embodiment of an exemplary system 10 is show for employing multi-stage processing to more efficiently process a large amount of data. The system 10 and an exemplary environment in which it may operate may broadly comprise a subject of monitoring 12,22,32; one or more sensors 14,24,34 generating the data; an electronic device 16,26,36 located locally relative to a subject of monitoring and performing initial processing of the data; a server computer 42 located remotely relative to the subject and performing final processing of the data; and a wireless communication network 44 allowing for bidirectional communication between the electronic device 16,26,36 and the server computer 42.

The subject of monitoring 12,22,32 may be substantially any subject for which monitoring is desired and possible. In an exemplary insurance implementation, the subject may be a vehicle 12, an item of property 22, or a person 32. The one or more sensors 14,24,34 may be substantially any suitable sensors configured or configurable to perform the desired monitoring. Examples of different sensors are discussed below. The electronic device 16,26,36 may be substantially any suitable device, such as a fixed or removable dedicated device or a smartphone, configured to receive the data from the one or more sensors 14,24,34, perform initial processing of the data, communicate the data to the server computer 42 via the communication network 44, receive a report from the server computer 42 via the communication network 44, and display the report for a user. The initial processing of the data may be performed by a software application 46 stored on and executed by the device 16,26,36, and may include identifying and transmitting any anomalous data to the server computer 42 in real time or near real time (e.g., within five minutes of identifying the anomalous data), storing or summarizing and storing any non-anomalous data locally, and transmitting the stored non-anomalous data to the server computer 42 at a later time.

In an exemplary driving insurance implementation, the one or more sensors 14 may detect anomalous driving-related behaviors which may include exceeding a speed limit, overly aggressive acceleration, turning, braking, cornering, and/or stopping, certain phone usage, driving and/or parking in high crime areas or, at least, outside of usual areas, non-use of a theft alarm, and/or a significant impact or a collision. The sensors 14 may further detect non-anomalous behaviors which may include speed, acceleration, turning, braking, cornering, stopping, certain phone usage, and/or location, and more generally, relevant behavior which does not qualify as anomalous. In this implementation, the electronic device 16 may be a dedicated device that plugs into the vehicle's OBD-II or other port, or the electronic device 16 may be the smartphone of the driver of the vehicle which is the subject of monitoring 12.

In an exemplary property insurance implementation, the one or more sensors 24 may detect anomalous home, business, or other real property safety- and/or security-related behavior which may include open or unlocked doors or windows, non-use of security lighting, and/or activation of thermal, audio, smoke, or security alarms. The sensors 24 may further detect non-anomalous behavior which, in general, may be relevant behavior which does not qualify as anomalous. In this implementation, the electronic device 26 may be a dedicated device fixedly mounted at or on the property which is the subject of monitoring 22.

In an exemplary health and/or life insurance implementation, the one or more sensors 34 may detect anomalous health- and/or life-related behavior which may include high resting heart rate and/or blood pressure, smoking, use of alcohol or other illegal drugs or certain legal drugs, and/or inadequate sleep. The sensors 34 may further detect non-anomalous behavior which may include heart rate and/or blood pressure, exercise, and/or sleep, and more generally, relevant behavior which does not qualify as anomalous. In this implementation, the electronic device 36 may be a smartphone carried by the person who is the subject of monitoring 32.

The server computer 42 may be substantially any suitable server configured to receive the anomalous data from the electronic device 16,26,36, perform final processing of the anomalous data, receive the non-anomalous data or a summary of the non-anomalous data at a later time, perform final processing of the non-anomalous data, generate a report of the anomalous and/or non-anomalous data, and communicate the report to the electronic device 16,26,36 via the communication network 44. Final processing of the anomalous and anon-anomalous data may be performed by software stored on and executed by the server computer 42, and may include scoring or otherwise reporting the anomalous data, scoring or otherwise reporting the non-anomalous data, and/or combining the anomalous and non-anomalous data and scoring or otherwise reporting the combined anomalous and non-anomalous data. In an exemplary insurance implementation, in which the large amount of data is relevant to assessing insurance risk, final processing may include recommending an insurance premium based on the scored or otherwise reported data.

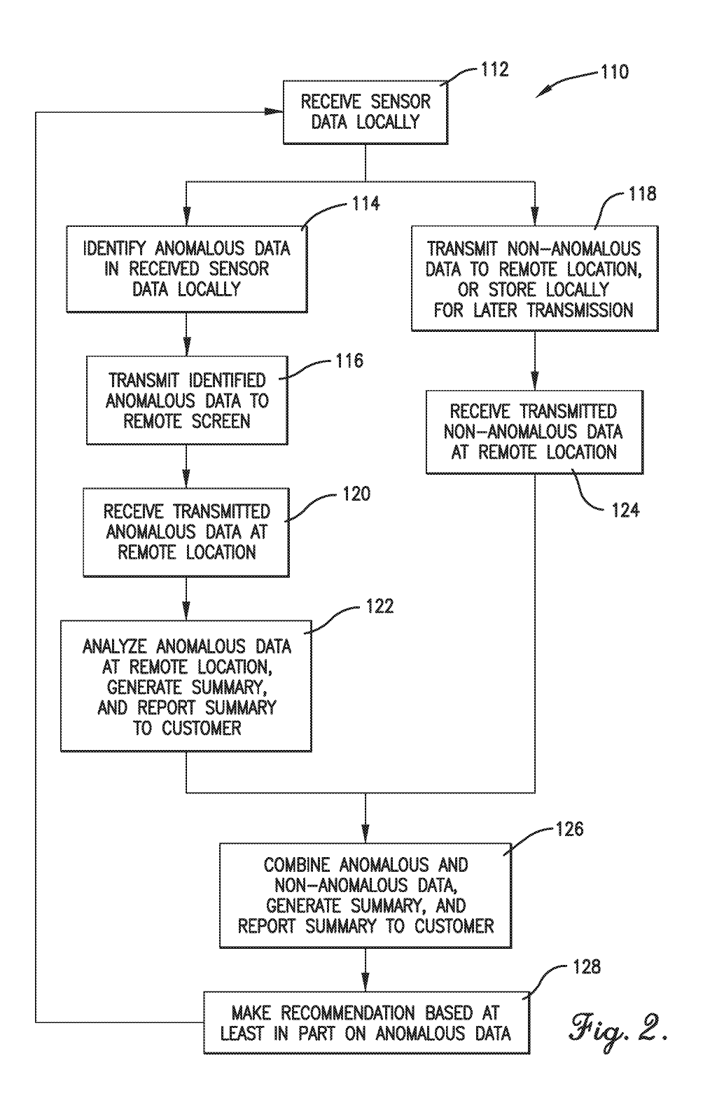

Referring to FIG. 2, the system 10 may function substantially as follows. The software application 46 executed by the local electronic device 16,26,36 may receive sensor data from the one or more sensors 14,24,34, as shown in 112. The software application 46 may perform initial processing of the data to identify anomalous data in the received sensor data, as shown in 114. The software application 46 may transmit the identified anomalous data to the server computer 42 via the communication network 44, as shown in 116. The anomalous data may be transmitted in real time or in near real time to the server computer 42. The software application 46 may transmit the non-anomalous data or a summary thereof to the server computer 42 also in real or near real time, or the software application 46 may store the non-anomalous data locally for later communication to the server computer 42 during an off-peak time, as shown in 118. Alternatively, the software application may discard or otherwise ignore the non-anomalous data if, for example, it is not deemed sufficiently valuable to transmit, in which case any score, report, decision, or recommendation may be based only on the anomalous data.

The server computer 42 may receive the transmitted anomalous data, as shown in 120. The server computer 42 may perform final processing of the anomalous data, generate a score or other report, and transmit the score or other report to the software application 46 for display for the user, as set forth in claim 122. The server computer 42 may receive the transmitted non-anomalous data or a summary thereof, as shown in 124. The server computer 42 may combine the anomalous and non-anomalous data, perform final processing of the combined data, generate a combined score or other report, and transmit the combined score or report to the software application 46 for display for the user, as shown in 126. In various alternative implementations, the server computer 42 may not combine the anomalous and non-anomalous data, or may combine the data but not generate a report of the combined data, or may generate a report of the combined data but not transmit the report for display to the user.

The server computer 42 may make a recommendation based, at least in part, on the analyzed anomalous data, as shown in 128. The recommendation may be based on the anomalous data or on the combined anomalous and non-anomalous data. The nature of the recommendation may depend on the implementation in which the system is being used. In an exemplary insurance implementation, the server computer 42 may recommend an insurance premium or other aspect of an insurance policy.

The system 10 may include more, fewer, or alternative components and/or perform more, fewer, or alternative actions, including those discussed elsewhere herein, and particularly those discussed in the following section describing the computer-implemented method.

Referring again to FIG. 2, an embodiment of a computer-implemented method 110 is shown for employing multi-stage processing to more efficiently process a large amount of data. The computer-implemented method 110 may be a corollary to the functionality of the system 10 of FIG. 1, and may be similarly implemented using the various components of the above-described system 10 within the exemplary operating environment.

The software application 46 executed by the local electronic device 16,26,36 may receive sensor data from the one or more sensors 14,24,34, as shown in 112. The software application 46 may perform initial processing of the data to identify anomalous data, or “segments of interest,” in the received sensor data, as shown in 114. The difference between anomalous and non-anomalous data may depend on the nature of the data and the implementation in which the system 10 is used, the needs and/or desires of the entity collecting the data, and/or applicable governmental regulations. Further, the difference may be objectively determined (e.g., behavior that exceeds a generally applicable threshold) and/or subjectively determined (e.g., behavior that exceeds a customer- or subject-specific threshold). Machine learning or other artificial intelligence techniques may be used to learn normal patterns of behavior in order to identify or better identify anomalous, or non-normal, behaviors. The definitions of anomalous data may be stored locally, such as on the electronic device 16,26,36, and may be updated as appropriate.

The software application 46 may transmit the anomalous data to the server computer 42 via the communication network 44, as shown in 116. The anomalous data may be transmitted in real time (i.e., as it is identified) or in near real time (e.g., within five minutes of being identified) to the server computer 42. If the server computer 42 and/or the communication network 44 are operating below their maximum capacities, then the software application 46 may transmit the non-anomalous data or a summary thereof to the server computer 42 also in real or near real time. During peak times, when the server computer 42 and/or the communication network 44 are having difficulty handling the amount of incoming data, the software application 46 may store the non-anomalous data locally for later communication to the server computer 42 during an off-peak time, as shown in 118. Further, rather than transmitting or even storing all of the non-anomalous data, the software application may score or summarize the non-anomalous data prior to transmission or storage, thereby saving network connection, data use, data storage, and back-end processing time.

The server computer 42 may receive the transmitted anomalous data, as shown in 120. The server computer 42 may perform final processing of the anomalous data, generate a score or other report, and transmit the score or other report to the software application 46 for display for the user, as set forth in claim 122. The nature of the final processing of the anomalous data may depend on the implementation in which the system is being used. In an exemplary insurance implementation, the anomalous data may be used to generate an insurance score. The score or other report of the anomalous data may be the only score or report communicated back to the user of the electronic device 16,26,36. Alternatively, it may be preliminary, and as discussed below, a comprehensive score or other report based on the combined anomalous and non-anomalous data may be transmitted later. The score or other report may be generated periodically (e.g., after each operation or use, daily, weekly, monthly, or quarterly) or continuously.

The server computer 42 may receive the transmitted non-anomalous data or a summary thereof, as shown in 124. The non-anomalous data may be transmitted by the software application 46 and received by the server computer 42 at a variable or fixed intervals, or as discussed, during off-peak or other more convenient times. The server computer 42 may combine the anomalous and non-anomalous data, perform final processing of the combined data, generate a combined score or other report, and transmit the combined score or report to the software application 46 for display for the user, as shown in 126. In various alternative implementations, the server computer 42 may not combine the anomalous and non-anomalous data, or may combine the data but not generate a report of the combined data, or may generate a report of the combined data but not transmit the report for display to the user.

The server computer 42 may make a recommendation based, at least in part, on the analyzed anomalous data, as shown in 128. In one implementation, the recommendation may be based on the combined anomalous and non-anomalous data. The nature of the recommendation may depend on the implementation in which the system is being used. In an exemplary insurance implementation, the server computer 42 may recommend an insurance premium.

In one implementation, the user may be allowed to annotate the data or append to it.

In one implementation, in order to further decrease the response time for customers, an estimation technique may be used prior to full analysis of the anomalous data. The technique may include providing estimations based on historical trends and future predictions that are then later reconciled with the actual processed data after to refine the results/future estimations. Exemplary applications for the estimation technique could include determining driving scores based on a number/frequency of anomalies before they are processed, thereby allowing for closer-to-real time feedback, and/or determining discount amounts for driving behavior discount models.

In one implementation, all or aspects of the process may be repeated periodically or continuously.

The computer-implemented method 110 may include more, fewer, or alternative actions, including those discussed elsewhere herein.

The present technology's use of multi-stage processing provides several advantages, including making more efficient use of resources and reducing delays in reporting at least the summaries of anomalous data to users. In particular, embodiments may transmit less data, which lowers data usage and battery consumption, provides better user experiences, reduces workload on the back-end, and allows users to see at least preliminary results in real or near real time.

Although the invention has been described with reference to the one or more embodiments illustrated in the figures, it is understood that equivalents may be employed and substitutions made herein without departing from the scope of the invention as recited in the claims.

Having thus described one or more embodiments of the invention, what is claimed as new and desired to be protected by Letters Patent includes the following:

We claim:

1. A system for collecting and processing information relevant to setting an insurance premium for a health or life of a person, the system comprising:
a local electronic device storing and executing a software application configured to—
receive sensor data from one or more sensors located on or about the person,
identify anomalous data in the sensor data as the anomalous data is received, and
transmit the anomalous data as the anomalous data is identified; and
a remote server computer configured to—
receive the anomalous data transmitted by the software application,
analyze the anomalous data, and
recommend the insurance premium based at least in part on the anomalous data.

2. The system as set forth in claim 1, wherein the local electronic device is a smartphone carried by the person, and the anomalous data is transmitted over a wireless communication network to the remote server computer.

3. The system as set forth in claim 1, wherein the sensor data includes heart rate and blood pressure, smoking, alcohol and drugs, exercise, and sleep.

4. The system as set forth in claim 3, wherein the anomalous data includes high resting heart rate and blood pressure, smoking; use of alcohol or other illegal drugs or certain legal drugs, and inadequate sleep.

5. The system as set forth in claim 1, wherein the anomalous data is defined objectively.

6. The system as set forth in claim 1, wherein the anomalous data is defined subjectively.

7. The system as set forth in claim 1, wherein an artificial intelligence tool is used to identify the anomalous data.

8. The system as set forth in claim 1, the server computer being further configured to generate a report based on the anomalous data, wherein the report includes a score, and to transmit the report to the local electronic device for display.

9. The system as set forth in claim 8, wherein the report is generated daily, weekly, monthly, or quarterly.

10. The system as set forth in claim 1, the software application being further configured to store non-anomalous data or a summary thereof, and to transmit the non-anomalous data or the summary thereof at an off-peak time.

11. The system as set forth in claim 10, the remote server computer being further configured to—
receive and analyze the non-anomalous data or the summary thereof,
generate a report based on the anomalous data and the non-anomalous data or the summary thereof, wherein the report includes a score, and to transmit the report to the local electronic device for display, and
recommend the insurance premium based on both the anomalous data and the non-anomalous data or the summary thereof.

12. The system as set forth in claim 11, wherein the report is generated daily, weekly, monthly, or quarterly.

13. A system for collecting and processing information relevant to setting an insurance premium for a health or life of a person, the system comprising:
a local electronic device storing and executing a software application configured to—
receive sensor data from one or more sensors located on or about the property, wherein the sensor data includes heart rate and blood pressure, smoking, alcohol and drugs, exercise, and sleep,
identify anomalous data in the sensor data as the anomalous data is received, wherein the anomalous data includes high resting heart rate and blood pressure, smoking; use of alcohol or other illegal drugs or certain legal drugs, and inadequate sleep,
transmit the anomalous data as the anomalous data is identified, and
store non-anomalous data and transmit the non-anomalous data at a later time; and
a remote server computer configured to—
receive the anomalous data and the non-anomalous data transmitted by the software application,
analyze the anomalous data and the non-anomalous data,
generate a report based on the anomalous data and the non-anomalous data, wherein the report includes a score, and transmit the report to the local electronic device for display, and
recommend the insurance premium based on both the anomalous data and the non-anomalous data.

14. A computer-implemented method for collecting and processing information relevant to setting an insurance premium for a health or life of a person, the computer-implemented method comprising:
receiving by a local electronic device sensor data from one or more sensors located on or about the person;
identifying by the local electronic device anomalous data in the sensor data as the anomalous data is received;
transmitting by the local electronic device the anomalous data as the anomalous data is identified;
receiving by a remote server computer the anomalous data transmitted by the local electronic device;
analyzing by the remote server computer the anomalous data, and
recommending by the remote server computer the insurance premium based at least in part on the anomalous data.

15. The computer-implemented method as set forth in claim 14, wherein the local electronic device is a smartphone carried by the person, and the anomalous data is transmitted over a wireless communication network to the remote server computer.

16. The computer-implemented method as set forth in claim 14, wherein the sensor data includes heart rate and blood pressure, smoking, alcohol and drugs, exercise, and sleep.

17. The computer-implemented method as set forth in claim 16, wherein the anomalous data includes high resting heart rate and blood pressure, smoking; use of alcohol or other illegal drugs or certain legal drugs, and inadequate sleep.

18. The computer-implemented method as set forth in claim 14, further including generating by the remote server computer a report based on the anomalous data, wherein the report includes a score, and transmitting the report to the local electronic device for display.

19. The computer-implemented method as set forth in claim 14, further including storing by the local electronic device non-anomalous data or a summary thereof, and transmitting the non-anomalous data or the summary thereof at an off-peak time.

20. The computer-implemented method as set forth in claim 19, further including—receiving and analyzing by the remote server computer the non-anomalous data or the summary thereof,
generating by the remote server computer a report based on the anomalous data and the non-anomalous data or the summary thereof, wherein the report includes a score, and transmitting the report to the local electronic device for display, and
recommending by the remote serve computer the insurance premium based on both the anomalous data and the non-anomalous data or the summary thereof.
